SEQUENCE LISTING (2 sequences)

SEQ ID NO: 1 - protein, 1272 aa, Artificial Sequence
  recombinant botulinum neurotoxin subtype E with reduced persistence with MDM2 binding motif
MPKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGSQDILLPNVIIMGAEPDLFETNSSNISLRNNYMPSNHGFGSIAIVTFSPEYSFRFNDNSMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLATKFQVKCRQTYIGQYKYFKLSNLLNDSIYNISEGYNINNLKVNFRGQNANLNPRIITPITGRGLVKKIIRFCVRGIITSLTFEHNWAQLENKSLVPRGSKALNDLCIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNAIKTIIESKYNSYTLEEKNELTNKYDIKQIENELNQKVSIAMNNIDRFLTESSISYLMKLINEVKINKLREYDENVKTYLLNYIIQHGSILGESQQELNSMVTDTLNNSIPFKLSSYTDDKILISYFNKFFKRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTLQDNAGINQKLAFNYGNANGISDYINKWIFVTITNDRLGDSKLYINGNLIDQKSILNLGNIHVSDNILFKIVNCSYTRYIGIRYFNIFDKELDETEIQTLYSNEPNTNILKDFWGNYLLYDKEYYLLNVLKPNNFIDRRKDSTLSINNIRSTILLANRLYSGIKVKIQRVNNSSTNDNLVRKNDQVYINFVASKTHLFPLYADTATTNKEKTIKISSSGNRFNQVVVMNSVGNNCTMNFKNNNGNNIGLLGFKADTVVASTWYYTHMRDHTNSNGCFWNFISEEHGWQEK

SEQ ID NO: 2 - protein, 1272 aa, Artificial Sequence
  botulinum neurotoxin subtype E with reduced persistence
MPKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGSQDILLPNVIIMGAEPDLFETNSSNISLRNNYMPSNHGFGSIAIVTFSPEYSFRFNDNSMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLATKFQVKCRQTYIGQYKYFKLSNLLNDSIYNISEGYNINNLKVNFRGQNANLNPRIITPITGRGLVKKIIRFCVRGIITSLTFEHNWAQLTSKSLVPRGSKALNDLCIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNAIKTIIESKYNSYTLEEKNELTNKYDIKQIENELNQKVSIAMNNIDRFLTESSISYLMKLINEVKINKLREYDENVKTYLLNYIIQHGSILGESQQELNSMVTDTLNNSIPFKLSSYTDDKILISYFNKFFKRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTLQDNAGINQKLAFNYGNANGISDYINKWIFVTITNDRLGDSKLYINGNLIDQKSILNLGNIHVSDNILFKIVNCSYTRYIGIRYFNIFDKELDETEIQTLYSNEPNTNILKDFWGNYLLYDKEYYLLNVLKPNNFIDRRKDSTLSINNIRSTILLANRLYSGIKVKIQRVNNSSTNDNLVRKNDQVYINFVASKTHLFPLYADTATTNKEKTIKISSSGNRFNQVVVMNSVGNNCTMNFKNNNGNNIGLLGFKADTVVASTWYYTHMRDHTNSNGCFWNFISEEHGWQEK

(19) United States
(12) Patent Application Publication
(10) Pub. No.: US 2017/0119863 A1
(43) Pub. Date: May 4, 2017

(54) NOVEL USES OF RECOMBINANT CLOSTRIDIAL NEUROTOXINS WITH DECREASED DURATION OF EFFECT

(71) Applicant: MERZ PHARMA GMBH & CO. KGAA, Frankfurt am Main (DE)

(72) Inventors: Klaus FINK, Köln (DE); Susanne GRAFE, Düsseldorf (DE); Imke SCHULTZ, Rodgau (DE); Susanna ROLL, Stadecken-Elsheim (DE)

(21) Appl. No.: 15/317,108

(22) PCT Filed: Jun. 12, 2015
(86) PCT No.: PCT/EP2015/001190

§ 371 Date: Dec. 7, 2016

(30) Foreign Application Priority Data
Jun. 13, 2014 (EP) 14002052.0

(51) Int. Cl. A61K 38/48; C12N 9/52

(52) U.S. Cl. CPC A61K 38/4893; C12Y 304/24069; C12N 9/52

(57) ABSTRACT

This invention relates to novel uses of recombinant clostridial neurotoxins exhibiting decreased duration of effect, in particular uses for the treatment of patients having experienced tendon-related disorders and/or injuries.

### FIELD OF THE INVENTION

This invention relates to novel uses of recombinant clostridial neurotoxins exhibiting decreased duration of effect, in particular uses for the treatment of patients having experienced a tendon-related disorders and/or injuries.

### BACKGROUND OF THE INVENTION

Clostridium is a genus of anaerobe gram-positive bacteria, belonging to the Firmicutes. Clostridium consists of around 100 species that include common free-living bacteria as well as important pathogens, such as Clostridium botulinum and Clostridium tetani. Both species produce neurotoxins, botulinum toxin and tetanus toxin, respectively. These neurotoxins are potent inhibitors of calcium-dependent neurotransmitter secretion of neuronal cells and are among the strongest toxins known to man. The lethal dose in humans lies between 0.1 ng and 1 ng per kilogram of body weight.

Oral ingestion of botulinum toxin via contaminated food or generation of botulinum toxin in wounds can cause botulism, which is characterised by paralysis of various muscles. Paralysis of the breathing muscles can cause death of the affected individual.

Although both botulinum neurotoxin (BoNT) and tetanus neurotoxin (TeNT) function via a similar initial physiological mechanism of action, inhibiting neurotransmitter release from the axon of the affected neuron into the synapse, they differ in their clinical response. While the botulinum toxin acts at the neuromuscular junction and other cholinergic synapses in the peripheral nervous system, inhibiting the release of the neurotransmitter acetylcholine and thereby causing flaccid paralysis, the tetanus toxin, which is transcytosed into central neurons, acts mainly in the central nervous system, preventing the release of the inhibitory neurotransmitters GABA (gamma-aminobutyric acid) and glycine by degrading the protein synaptobrevin. The consequent overactivity of spinal cord motor neurons causes generalized contractions of the agonist and antagonist musculature, termed a tetanic spasm (rigid paralysis).

While the tetanus neurotoxin exists in one immunologically distinct type, the botulinum neurotoxins are known to occur in seven different immunogenic serotypes, termed BoNT/A through BoNT/H with further subtypes. Most Clostridium botulinum strains produce one type of neurotoxin, but strains producing multiple toxins have also been described.

Botulinum and tetanus neurotoxins have highly homologous amino acid sequences and show a similar domain structure. Their biologically active form comprises two peptide chains, a light chain of about 50 kDa and a heavy chain of about 100 kDa, linked by a disulfide bond. A linker or loop region, whose length varies among different clostridial toxins, is located between the two cysteine residues forming the disulfide bond. This loop region is proteolytically cleaved by an unknown clostridial endoprotease to obtain the biologically active toxin.

The molecular mechanism of intoxication by TeNT and BoNT appears to be similar as well: entry into the target neuron is mediated by binding of the C-terminal part of the heavy chain to a specific cell surface receptor; the toxin is then taken up by receptor-mediated endocytosis. The low pH in the so formed endosome then triggers a conformational change in the clostridial toxin which allows it to embed itself in the endosomal membrane and to translocate through the endosomal membrane into the cytoplasm, where the disulfide bond joining the heavy and the light chain is reduced. The light chain can then selectively cleave so called SNARE-proteins, which are essential for different steps of neurotransmitter release into the synaptic cleft, e.g. recognition, docking and fusion of neurotransmitter-containing vesicles with the plasma membrane. TeNT, BoNT/B, BoNT/D, BoNT/F, and BoNT/G cause proteolytic cleavage of synaptobrevin or VAMP (vesicle-associated membrane protein), BoNT/A and BoNT/E cleave the plasma membrane-associated protein SNAP-25, and BoNT/C cleaves the integral plasma membrane protein syntaxin and SNAP-25.

In Clostridium botulinum, the botulinum toxin is formed as a protein complex comprising the neurotoxic component and non-toxic proteins. The accessory proteins embed the neurotoxic component thereby protecting it from degradation by digestive enzymes in the gastrointestinal tract. Thus, botulinum neurotoxins of most serotypes are orally toxic. Complexes with, for example, 450 kDa or with 900 kDa are obtainable from cultures of Clostridium botulinum.

In recent years, botulinum neurotoxins have been used as therapeutic agents, for example in the treatment of dystonias and spasms, and have additionally been used in cosmetic applications, such as the treatment of fine wrinkles. Preparations comprising botulinum toxin complexes are commercially available, e.g. from Ipsen Ltd (Dysport®) or Allergan Inc. (Botox®). A high purity neurotoxic component, free of any complexing proteins, is for example available from Merz Pharmaceuticals GmbH, Frankfurt (Xeomin®).

Clostridial neurotoxins are usually injected into the affected muscle tissue, bringing the agent close to the neuromuscular end plate, i.e. close to the cellular receptor mediating its uptake into the nerve cell controlling said affected muscle. Various degrees of neurotoxin spread have been observed. The neurotoxin spread is thought to depend on the injected amount and the particular neurotoxin preparation. It can result in adverse side effects such as paralysis in nearby muscle tissue, which can largely be avoided by reducing the injected doses to the therapeutically relevant level. Overdosing can also trigger the immune system to generate neutralizing antibodies that inactivate the neurotoxin preventing it from relieving the involuntary muscle activity. Immunologic tolerance to botulinum toxin has been shown to correlate with cumulative doses.

Clostridial neurotoxins display variable durations of action that are serotype specific. The clinical therapeutic effect of BoNT/A lasts approximately 3 months for neuromuscular disorders and 6 to 12 months for hyperhidrosis. The effects of BoNT/E, on the other hand, last about 4 weeks. One possible explanation for the divergent durations of action might be the distinct subcellular localizations of BoNT serotypes. The protease domain of BoNT/A light chain localizes in a punctate manner to the plasma membrane of neuronal cells, co-localizing with its substrate SNAP-25. In contrast, the short-duration BoNT/E serotype is cytoplasmic. Membrane association might protect BoNT/A from cytosolic degradation mechanisms allowing for prolonged persistence of BoNT/A in the neuronal cell.

The longer lasting therapeutic effect of BoNT/A makes it preferable for certain clinical uses and in particular for certain cosmetic uses compared to the other serotypes, for example serotypes B, C₁, D, E, F, G and H. On the other hand, it might be advantageous in certain scenarios to decrease the duration of the therapeutic effect of a botulinum neurotoxin in order to reduce the duration of muscle paralysis.

WO 2011/000929 and WO 2013/068476 describe neurotoxins exhibiting a shortened biological activity. In brief, the applications describe polypeptides comprising at least one E3 ligase recognition motif in the light chain, wherein said E3 ligase recognition motif is preferably a binding motif for the E3 ligase MDM2. Section [0006] of WO 2013/068476 generically lists a number of indications, which could potentially benefit from the application of modified neurotoxins with decreased duration of effect.

In particular, WO 2013/068476 describes variants of BoNT/E (SEQ ID NOs: 52 and 80 in WO 2013/068476), which were shown to have a duration of effect, which was decreased by about 25% compared to wild-type BoNT/E in a cell culture assay.

Despite the progress that has been made in the past in the treatment of indications that benefit from the intermittent paralysis of muscles, there is still a strong demand to further improve the therapeutic options available to the practitioner in the art, in particular in light of the fact that it might be desirable in certain indications, after an initial requirement for paralysing one or more muscles in such indication, to achieve an earlier recovery of muscle activity to assist the patient being treated in getting back to his or her normal life. To date, such aspects have not been addressed satisfactorily.

### OBJECTS OF THE INVENTION

It was an object of the invention to provide novel uses for recombinant clostridial neurotoxins exhibiting a decreased duration of effect, and to improve the treatment of patients having experienced a tendon-related disorders and/or injuries, in particular rotator cuff tear or retear, biceps tendon tear or retear, Achilles tendon tear or retear, tears or retears of a flexor or extensor tendon of the underarm, hand or finger, or a tendinopathy, such as epicondylitis.

### SUMMARY OF THE INVENTION

The naturally occurring botulinum toxin serotypes display highly divergent durations of effect, with BoNT/A exhibiting the longest persistence, and BoNT/E exhibiting a comparatively short persistence. In order to broaden the applicability of botulinum neurotoxins, variants of BoNT/E have been created that exhibit a shorter duration of effect (see in particular WO 2013/068476).

Surprisingly, it has been identified that the variants disclosed in WO 2013/068476 might advantageously be used in particular situations, for which no satisfactory solution has been available so far.

Thus, the present invention relates to a botulinum neurotoxin subtype E with reduced persistence having a sequence according to SEQ ID NO: 1, or SEQ ID NO: 2, or a functionally active variant thereof, for use in the treatment of a patient suffering from a tendon-related disorder and/or injury.

In a second aspect, the present invention relates to a method for the treatment of a patient suffering from a tendon-related disorder and/or injury, comprising the step of administering a botulinum neurotoxin subtype E with reduced persistence having a sequence according to SEQ ID NO: 1, or SEQ ID NO: 2, or a functionally active variant thereof, to said patient.

### DETAILED DESCRIPTION OF THE INVENTION

The present invention may be understood more readily by reference to the following detailed description of the invention and the examples included therein.

Thus, the present invention relates to a botulinum neurotoxin subtype E with reduced persistence having a sequence according to SEQ ID NO: 1, or SEQ ID NO: 2 or a functionally active variant thereof, for use in the treatment of a patient suffering from a tendon-related disorder and/or injury.

In a second aspect, the present invention relates to a method for the treatment of a patient suffering from a tendon-related disorder and/or injury, comprising the step of administering a botulinum neurotoxin subtype E with reduced persistence having a sequence according to SEQ ID NO: 1, or SEQ ID NO: 2, or a functionally active variant thereof, to said patient.

In the context of the present invention, the term “functionally active variant” refers to a neurotoxin, in particular a recombinant neurotoxin, that differs in the amino acid sequence and/or the nucleic acid sequence encoding the amino acid sequence from the botulinum neurotoxin subtype E with reduced persistence having a sequence according to SEQ ID NO: 1 or SEQ ID NO: 2, but is still functionally active. In the context of the present invention, the term “functionally active” refers to the property of such recombinant clostridial neurotoxin variant to (i) achieve muscle paralysis to at least 50%, particularly to at least 60%, at least 70%, at least 80%, and most particularly at least 90% of the muscle paralysis achieved with the same amount of a botulinum neurotoxin subtype E with reduced persistence having a sequence according to SEQ ID NO: 1 or SEQ ID NO: 2, and (ii) achieve such muscle paralysis for a duration of time that is at maximum 10% shorter or longer, particularly at maximum 5% shorter or longer than the duration of paralysis achieved by a botulinum neurotoxin subtype E with reduced persistence having a sequence according to SEQ ID NO: 1 or SEQ ID NO: 2 (i.e. which shows between 90% and 110% of the duration of paralysis, particularly between 95% and 105% of the duration of paralysis achieved by a botulinum neurotoxin subtype E with reduced persistence having a sequence according to SEQ ID NO: 1 or SEQ ID NO: 2).

On the protein level, a functionally active variant will maintain key features of the corresponding parental clostridial neurotoxin, such as key residues for the endopeptidase activity in the light chain, or key residues for the attachment to the neurotoxin receptors or for translocation through the endosomal membrane in the heavy chain, but may contain one or more mutations comprising a deletion of one or more amino acids of the corresponding clostridial neurotoxin, an addition of one or more amino acids of the corresponding clostridial neurotoxin, and/or a substitution of one or more amino acids of the corresponding clostridial neurotoxin. Particularly, said deleted, added and/or substituted amino acids are consecutive amino acids. According to the teaching of the present invention, any number of amino acids may be added, deleted, and/or substituted, as long as the functionally active variant remains biologically active as defined above. For example, 1, 2, 3, 4, 5, up to 10, up to 15, up to 25, up to 50, up to 100, up to 200, up to 400, up to 500 amino acids or even more amino acids of a botulinum neurotoxin subtype E with reduced persistence having a sequence according to SEQ ID NO: 1 or SEQ ID NO: 2 may be added, deleted, and/or substituted. This neurotoxin fragment may contain an N-terminal, C-terminal, and/or one or more internal deletion(s).

In another embodiment, the functional variant of a clostridial neurotoxin additionally comprises a signal peptide. Usually, said signal peptide will be located at the N-terminus of the neurotoxin. Many such signal peptides are known in the art and are comprised by the present invention. In particular, the signal peptide results in transport of the neurotoxin across a biological membrane, such as the membrane of the endoplasmic reticulum, the Golgi membrane or the plasma membrane of a eukaryotic or prokaryotic cell. It has been found that signal peptides, when attached to the neurotoxin, will mediate secretion of the neurotoxin into the supernatant of the cells. In certain embodiments, the signal peptide will be cleaved off in the course of, or subsequent to, secretion, so that the secreted protein lacks the N-terminal signal peptide, is composed of separate light and heavy chains, which are covalently linked by disulfide bridges, and is proteolytically active.

In particular embodiments, the functional variant has in its clostridium neurotoxin part a sequence identity of at least 40%, at least 50%, at least 60%, at least 70% or most particularly at least 80%, and a sequence homology of at least 60%, at least 70%, at least 80%, at least 90%, or most particularly at least 95% to the corresponding part of a botulinum neurotoxin subtype E with reduced persistence having a sequence according to SEQ ID NO: 1 or SEQ ID NO: 2. Methods and algorithms for determining sequence identity and/or homology, including the comparison of variants having deletions, additions, and/or substitutions relative to a parental sequence, are well known to the practitioner of ordinary skill in the art. On the DNA level, the nucleic acid sequences encoding the functional homologue and the parental clostridial neurotoxin may differ to a larger extent due to the degeneracy of the genetic code. It is known that the usage of codons is different between prokaryotic and eukaryotic organisms. Thus, when expressing a prokaryotic protein such as a clostridial neurotoxin, in a eukaryotic expression system, it may be necessary, or at least helpful, to adapt the nucleic acid sequence to the codon usage of the expression host cell, meaning that sequence identity or homology may be rather low on the nucleic acid level.

In the context of the present invention, the term “variant” refers to a neurotoxin that is a chemically, enzymatically, or genetically modified derivative of a botulinum neurotoxin subtype E with reduced persistence having a sequence according to SEQ ID NO: 1 or SEQ ID NO: 2. A chemically modified derivative may be one that is modified by pyruvation, phosphorylation, sulfatation, lipidation, pegylation, glycosylation and/or the chemical addition of an amino acid or a polypeptide comprising between 2 and 100 amino acids, including modification occurring in the eukaryotic host cell used for expressing the derivative. An enzymatically modified derivative is one that is modified by the activity of enzymes, such as endo- or exoproteolytic enzymes, including modification by enzymes of the eukaryotic host cell used for expressing the derivative. As pointed out above, a genetically modified derivative is one that has been modified by deletion or substitution of one or more amino acids contained in, or by addition of one or more amino acids (including polypeptides comprising between 2 and about 100 amino acids) to, the amino acid sequence of said clostridial neurotoxin. Methods for designing and constructing such chemically or genetically modified derivatives and for testing of such variants for functionality are well known to anyone of ordinary skill in the art.

In the context of the present invention, the term “recombinant neurotoxin” refers to a composition comprising a clostridial neurotoxin that is obtained by expression of the neurotoxin in a heterologous cell such as E. coli, and including, but not limited to, the raw material obtained from a fermentation process (supernatant, composition after cell lysis), a fraction comprising a clostridial neurotoxin obtained from separating the ingredients of such a raw material in a purification process, an isolated and essentially pure protein, and a formulation for pharmaceutical and/or aesthetic use comprising a clostridial neurotoxin and additionally pharmaceutically acceptable solvents and/or excipients.

In the context of the present invention, the term “comprises” or “comprising” means “including, but not limited to”. The term is intended to be open-ended, to specify the presence of any stated features, elements, integers, steps or components, but not to preclude the presence or addition of one or more other features, elements, integers, steps, components, or groups thereof. The term “comprising” thus includes the more restrictive terms “consisting of” and “consisting essentially of”.

In the context of the present invention, the term “botulinum neurotoxin subtype E” refers to a particular neurotoxin found in and obtainable from Clostridium botulinum having a sequence shown in SEQ ID NO: 82 of WO 2013/068476.

In particular embodiments, said functionally active variant has a persistence that is at maximum 5% shorter or longer than the duration of paralysis achieved by a botulinum neurotoxin subtype E with reduced persistence having a sequence according to SEQ ID NO: 1 or SEQ ID NO: 2.

Without wishing to be bound by theory, the recombinant clostridial neurotoxins of the present invention might show decreased biological half-life, increased degradation rates, increased diffusion rates, decreased uptake by neuronal cells, and/or modified intracellular translocation rates, in each case relative to wild-type botulinum neurotoxin of subtype E (BoNT/E).

Tendon-related disorders and/or injuries may be caused by overuse, traumata, such as injuries, including microtears and complete tears, inflammatory conditions or use of corticosteroids. Tendon-related disorders and/or injuries are particularly difficult to treat since on the one hand tendons are not well vascularized so that blood supply is limited, and supply of nutrients has to occur via synovial liquid, and tendons are subject to strong forces or loads due to the activity of the muscles acting on or attached to the tendons. This is particularly relevant in the case of torn tendon that are surgically reconstructed and where a rather high risk of re-tears is seen in the first days or weeks after surgery.

Thus, in principle it would be desirable to immobilize that part of the body that normally exerts strong forces or load on the tendon to be treated in a tendon-related disorders and/or injuries, but such immobilization may result in the production of less collagen, in muscle and/or tendon atrophy and in particular in stiffening of the joint adjacent to the site of immoblilzation (see Leesa M. Galatz et al: Complete removal of load is detrimental to rotator cuff healing; Jomal of shoulder and elbow surgery (2009) 18, 669-675).

In particular embodiments, the tendon-related disorder and/or injury is selected from the list of: rotator cuff tear or retear, biceps tendon tear or retear, Achilles tendon tear or retear, tear or retear of a flexor or extensor tendon of the underarm, hand or finger, or a tendinopathy, such as epicondylitis.

In particular embodiments, the treatment includes a repair of a tendon tear by surgery.

In certain such embodiments, the treatment comprises the administration of the botulinum neurotoxin subtype E with reduced persistence having a sequence according to SEQ ID NO: 1, or SEQ ID NO: 2 or a functionally active variant thereof, after surgery (post-operatively) in order to immobilize one or more muscles that could exert forces or load on said tendon.

In particular other embodiments, the treatment comprises the administration of the botulinum neurotoxin subtype E with reduced persistence having a sequence according to SEQ ID NO: 1, or SEQ ID NO: 2 or a functionally active variant thereof, prior to surgery (pre-operatively) or during surgery (pen-operatively) in order to relax one or more muscles that exert forces or load on those parts of the tendon that have to be re-connected.

Due to the short-acting profile of botulinum neurotoxin subtype E with reduced persistence having a sequence according to SEQ ID NO: 1, or SEQ ID NO: 2 or a functionally active variant thereof, it becomes possible to re-start light physical activities earlier, which result in the formation of tendon material with better biomechanical properties due to the mechanical stimulation (see: Andersson et al. Low-level mechanical stimulation is sufficient to improve tendon healing in rats; J Appl Physiol 113L1398-1402, 2012; (Schepuli and Aspenberg: Early controlled tension improves the material properties of healing human Achilles tendons after ruptures: A randomized trial; The American Journal of Sports Medicine; 09/2013; K 41 (11).). Furthermore, the possibility to re-start light movements earlier prevents adhesion processes and improves the transport of the synovial liquids, which improves the supply with nutrients and thus the healing of the tendon. As a result of the treatment with such short-acting botulinum toxin, the production of collagen may be improved, and the formation of gaps between the repaired tendon and the bone may be reduced.

A rotator cuff tear is a tear of one or more of the tendons of the four rotator cuff muscles. A rotator cuff injury can include any type of irritation or damage to the rotator cuff muscles or tendons. Rotator cuff tears are among the most common conditions affecting the shoulder.

The tendons of the rotator cuff, not the muscles, are most commonly involved and of the four, the supraspinatus most frequently, as it passes below the acromion. Such a tear usually occurs at its point of insertion onto the humeral head at the greater tubercle.

The cuff is responsible for stabilizing the glenohumeral joint and rotating the humerus outward (external rotation). When shoulder trauma occurs, these functions can be compromised. Because individuals are highly dependent on the shoulder for many activities, overuse of the muscles can lead to tears, with the vast majority again occurring in the supraspinatus tendon.

Rotator cuff tears are diagnosed by ultrasound imaging as either partial or full thickness tears (complete), where the tendon has been completely separated in two.

Partial tears are often treated with physical therapy but do proceed to surgery if a satisfactory outcome is not achieved.

Complete tears are usually treated by surgical reattachment of the tendon. Grade 2 and higher tears exhibit greater than 2-3 cm of retraction of the tendon and are considered most difficult to treat and associated with a higher incidence of retearing.

In addition to retears, complete tears with considerable tendon retraction may not be able to be repaired surgically. Total shoulder replacement may be the only viable treatment option.

An estimated 76,000 rotator cuff repairs were performed in the seven major markets in 2008, based on the average procedure volume in the US from 1998-2005. Low level growth is expected in the US due to population growth and an aging population. Despite an extremely large number of physician visits being recorded for rotator cuff injuries, there are a relatively small number of rotator cuff repairs being performed.

Analysis of US hospital discharge statistics indicates that from 1996 to 2006, total RCR procedures increased over 140% with the majority being performed arthroscopically.

The purpose of rotator cuff repair is to diminish pain and restore function. This most predictably occurs when the tendon is demonstrated to heal.

Recovery after rotator cuff surgery is dependent upon multiple factors including the completeness of tear, patient performance, and age. The greatest concern for rotator cuff surgery patients is the potential for repeated injury. Retear following surgical repair results in decreased patient function and represents an unmet need, despite high levels of patient satisfaction with the initial outcomes of surgery. Retears are attributed to a variety of factors including muscle retraction which places tension on the repair.

In addition to retears, complete tears with considerable tendon retraction may not be able to be repaired surgically. In such cases, total shoulder replacement may be the only viable treatment option. Complete immobilization of the shoulder is required postoperatively.

A recently published study (in rats) indicated that tendon-to-bone healing after rotator cuff repair can be altered positively using botulinum toxin subtype A pre-operatively. Tears with increased repair load seem to benefit the most—at least histologically (Ficklscherer et al, J Orthop Res, 2013 May).

This was formerly detected in another study in rats (Hettrich et al, J Shoulder Elbow Surg. 2011). Botulinum toxin A-treated specimens had increased collagen fiber organization at 4 weeks and decreased mechanical properties at later time points. Longlasting reduction in muscle loading and contraction resulted in delayed and reduced bone mineralization and tendon insertion in animal experiments (Galatz et al. 2009 J Shoulder Elbow Surg 18:669-675; Thomopoulos et al. 2007 J Orthop Res 25:1154-1163).

However, the long-lasting effect of BoNT/A, and even the shorter effect of wild-type BoNT/E, appear to be not permitting their use in patients, since it is believed that long-term immobilization of muscles involved in movement of the shoulder may ultimately lead to muscle atrophy and in particular to stiffening of the shoulder.

The proteins having a sequence according to SEQ ID NO: 1 or SEQ ID NO: 2 are local muscle relaxants with an onset of effect within a day and an estimated duration of effect of 4 weeks (+2 weeks). An injection of one of these proteins reduces the rate of retears and additionally post-surgical pain, which has been attributed in part to the tension caused by muscle retraction. The advantages of such treatment are:

Pre-operative administration a protein having a sequence according to SEQ ID NO: 1 or SEQ ID NO: 2 will facilitate surgical repair by causing the tendon/muscle unit to be more pliable. This pliability would allow the surgeon to more easily repair retracted tendons found in older tears and in complete tears that are grade 2 and beyond.

The increased pliability of the muscle tendon unit and the reduced tension on repaired rotator cuff tendons will allow an increased opportunity to heal and will ultimately reduce the rate of retears.

Complete immobilization during the first two weeks of recovery is part of a standard recovery program. This timeframe is completely covered by the effects of a protein having a sequence according to SEQ ID NO: 1 or SEQ ID NO: 2 and would support the healing process.

By four weeks post-op, patients will begin passive range of motion exercises. This is in line with the end of the paralyzing effect of a protein having a sequence according to SEQ ID NO: 1 or SEQ ID NO: 2.

In addition, post-surgical pain which has been attributed in part to the tension caused by muscle retraction may be reduced.

Finally, the duration of botulinum toxin specific side effects will be reduced due to the shorter duration of the toxin. Additionally atrophy of the injected muscles will be avoided in contrast to a longer acting botulinum toxin.

In particular embodiments, said treatment comprises the improved healing of a rotator cuff condition selected from the list of: (a) rotator cuff with partial tear of one or more of the supraspinatus, infraspinatus, deltoideus, teres minor, teres major, and/or subscapularis tendon(s) only, (b) rotator cuff with partial or complete tear of the tendinous and/or muscular part of one or more muscles selected from the list of: supraspinatus, infraspinatus, teres minor, and subscapularis; (c) rotator cuff with complete tear of one or more of the supraspinatus, infraspinatus, teres minor, and/or subscapularis tendon(s) only.

In particular embodiments, said treatment comprises the improved healing of one or more of the supraspinatus, infraspinatus, teres minor, and/or subscapularis tendon(s).

In particular embodiments, said treatment further comprises the improved healing of one or more muscles selected from the list of supraspinatus, infraspinatus, teres minor, and subscapularis.

In particular embodiments, said treatment further comprises the improved healing of one or more muscles selected from the list of: deltoideus and teres major.

In particular embodiments, said treatment comprises the administration of said botulinum neurotoxin subtype E with reduced persistence having a sequence according to SEQ ID NO: 1 or SEQ ID NO: 2, or of said functionally active variant thereof, to one or more muscles selected from the list of: supraspinatus, infraspinatus, teres minor, and subscapularis

In particular embodiments, muscle paralysis by a botulinum neurotoxin of more than 5 weeks, in particular of more than 4 weeks, and more particularly of more than 3 weeks, is contraindicated and/or deemed to be associated with negative impact on overall treatment success, particularly due to high likelihood of increased muscle atrophy.

In particular embodiments, the condition is biceps tendon tear or retear. In particular such embodiments, the treatment comprises the administration of said botulinum neurotoxin subtype E with reduced persistence having a sequence according to SEQ ID NO: 1 or SEQ ID NO: 2, or of said functionally active variant thereof, to one or more muscles selected from the list of: biceps and triceps.

In particular embodiments, the condition is Achilles tendon tear or retear. In particular such embodiments, the treatment comprises the administration of said botulinum neurotoxin subtype E with reduced persistence having a sequence according to SEQ ID NO: 1 or SEQ ID NO: 2, or of said functionally active variant thereof, to one or more muscles selected from the list of: M. gastrocnemius, soleus and plantaris.

In particular embodiments, the condition is peroneus and tibialis tendon injuries and tears or retear.

In particular embodiments, the condition is a tear or retear of a flexor or extensor tendon of the underarm, hand or finger. In particular such embodiments, the treatment comprises the administration of said botulinum neurotoxin subtype E with reduced persistence having a sequence according to SEQ ID NO: 1 or SEQ ID NO: 2, or of said functionally active variant thereof, to one or more muscles selected from the list of: flexor digitorum superficialis, flexor pollicis longus, flexor carpi ulnaris, flexor carpi radialis, flexor digitorum profundus, flexor digitorum sublimis, extensor policis, extensor carpi radialis, extensor digitorum, extensor digiti minimi, extensor arpi ulnaris, and extensor indicis.

Tendinopathy is an acquired non-inflammatory degeneration of tendons initially caused by irritation or overuse of tendons and microtears. Such degenerations may result in inflammatory conditions, which cause pain. Short-term muscle relaxation in proximity to a tendon with tendinopathy prevents repetitive microtrauma of the tendinous fibers at their origin and thereby allows healing process, thus relieving the patient's pain and assisting in the treatment of the inflammatory condition. Any tendon and its surrounding tissue can undergo a tendinopathic process. Certain tendons are particularly vulnerable to degenerative pathology; these include but are not limited to: wrist extensors (lateral epicondylitis), wrist flexors (medial epicondylitis), extensor pollicis brevis and abductor pollicis longus (Quervain disease), Achilles tendon, patella tendon, elements of the rotator cuff, forearm extensors, biceps brachii and tibialis posterior, peroneus, biceps femoris, semitendinousus, semimemranosus, quadriceps. Disorders of these tendons are often chronic and can be difficult to manage successfully in the long term.

In particular embodiments, the treatment comprises the administration of said botulinum neurotoxin subtype E with reduced persistence having a sequence according to SEQ ID NO: 1 or SEQ ID NO: 2, or of said functionally active variant thereof, to one or more muscles selected from the list of: ischiocrural muscles (hamstring muscles), M. biceps femoris, semitendinosus, semimembranosus.

In particular embodiments, the tendinopathy condition is epicondylitis.

Epicondylitis is an acquired non-inflammatory state of irritation of tendons at sites proximal to the a tendon base and is often caused by overuse of muscles, particularly of non-trained muscles, or caused by non-physiological movements, including epicondylitis humeri radialis (tennis elbow) and epicondylitis humeri ulnaris (golf elbow).

In particular embodiments, the condition is epicondylitis humeri radialis. In particular such embodiments, the treatment comprises the administration of said botulinum neurotoxin subtype E with reduced persistence having a sequence according to SEQ ID NO: 1 or SEQ ID NO: 2, or of said functionally active variant thereof, to one or more muscles selected from the list of: musculus extensor digitorum communis, musculus extensor carpi radialis brevis, M. extensor carpi ulnaris, and M. extensor carpi radialis longus.

In particular embodiments, the condition is epicondylitis ulnaris humeri. In particular such embodiments, the treatment comprises the administration of said botulinum neurotoxin subtype E with reduced persistence having a sequence according to SEQ ID NO: 1 or SEQ ID NO: 2, or of said functionally active variant thereof, to one or more muscles selected from the list of: musculus flexor digitorum profundus, M. pronator teres, M. flexor carpi ulnaris, M. palmaris longus, and M. flexor carpi radialis.

### EXAMPLES

### Example 1

### Treatment of Rotator Cuff Tear

A patient undergoes a rotator cuff repair after an injury. During the surgery, botulinum neurotoxin subtype E with reduced persistence having a sequence according to SEQ ID NO: 1 is injected in both the supraspinatus muscle and the infraspinatus muscle. The shoulder is completely immobilized for two weeks. Then the patient starts with passive movements. Range of motion QoL and Pain improves within weeks after surgery, and even one year after surgery the patient does not suffer a retear.

### Example 2

### Design of Clinical Trial

A phase 1 dose-response using an accepted model by the Regulatory Agencies study is foreseen because classical PK phase 1 studies are not possible with botulinum neurotoxins. Dose-response profile and duration of effect as well as systemic diffusion in adjacent muscles after a single intramuscular injection of modified Botulinum Neurotoxin subtype E with reduced persistence having a sequence according to SEQ ID NO: 1 in 3 to 4 concentrations into the Extensor Digitorum brevis (EDB) muscle will be investigated in healthy male volunteers in a single center, double-blind randomized study.

The planned observation period is up to 12 weeks after injection. Study parameters are the EDB-Compound Muscle Action Potential (CMAP) M-wave amplitude, Abductor hallucis-CMAP M-wave amplitude, Abductor digiti quinti-CMAP M-wave amplitude, and Adverse Events.

The investigation of safety, tolerability and efficacy of Fastox in rotator cuff repair is aim of a Phase 2a study with a randomized, double-blind, placebo-controlled, parallel group design. Up to 60 patients will be injected with modified Botulinum Neurotoxin subtype E with reduced persistence having a sequence according to SEQ ID NO: 1. Incidence of Retear. Range of Motion (ROM), Pain Scale and QoL Assessments, and amount of physiotherapy will be investigated 2, 6, 12, 18, and 24 weeks after injection.

1. A botulinum neurotoxin subtype E with reduced persistence having a sequence according to SEQ ID NO: 1, or SEQ ID NO: 2 or a functionally active variant thereof, for use in the treatment of a patient having experienced a tendon-related disorder and/or injury.

2. The botulinum neurotoxin subtype E with reduced persistence for use in the treatment according to claim 1, wherein said botulinum neurotoxin subtype E with reduced persistence is a functionally active variant of a sequence according to SEQ ID NO: 1, or SEQ ID NO: 2, wherein said functionally active variant has a persistence that is at maximum 5% shorter or longer than the duration of paralysis achieved by a botulinum neurotoxin subtype E with reduced persistence having a sequence according to SEQ ID NO: 1 or SEQ ID NO: 2.

3. The botulinum neurotoxin subtype E with reduced persistence for use in the treatment of claim 1, wherein said tendon-related disorder and/or injury is selected from the list of: rotator cuff tear or retear, biceps tendon tear or retear, Achilles tendon tear or retear, tear of the flexor or extensor tendon of the underarm, hand or finger, and a tendinopathy, particularly optionally epicondylitis.

4. The botulinum neurotoxin subtype E with reduced persistence for use in the treatment of claim 3, wherein said tendon-related disorder and/or injury is a rotator cuff tear or retear.

5. The botulinum neurotoxin subtype E with reduced persistence for use in the treatment of claim 4, wherein said tendon-related disorder and/or injury is a rotator cuff tear or retear selected from the list of: (a) rotator cuff with partial tear of one or more of the supraspinatus, infraspinatus, teres minor, and/or subscapularis tendon(s) only, (b) rotator cuff with partial or complete tear of the tendinous and/or muscular part of one or more muscles selected from the list of: supraspinatus, infraspinatus, teres minor, and subscapularis; and (c) rotator cuff with complete tear of one or more of the supraspinatus, infraspinatus, teres minor, and/or subscapularis tendon(s) only.

6. The botulinum neurotoxin subtype E with reduced persistence for use in the treatment of claim 4, wherein said treatment comprises the improved healing of one or more of the supraspinatus, infraspinatus, teres minor, and/or subscapularis tendon(s).

7. The botulinum neurotoxin subtype E with reduced persistence of claim 6, wherein said treatment further comprises the improved healing of one or more muscles selected from the list of supraspinatus, infraspinatus, teres minor, and subscapularis.

8. The botulinum neurotoxin subtype E with reduced persistence for use in the treatment of claim 4, wherein said treatment comprises the administration of said botulinum neurotoxin subtype E with reduced persistence having a sequence according to SEQ ID NO: 1 or SEQ ID NO: 2, or of said functionally active variant thereof, to one or more muscles selected from the list of: supraspinatus, infraspinatus, teres minor, and subscapularis.

9. The botulinum neurotoxin subtype E with reduced persistence for use in the treatment of claim 4, wherein muscle paralysis by a botulinum neurotoxin of more than 5 weeks, optionally of more than 4 weeks, and optionally of more than 3 weeks, is contraindicated and/or deemed to be associated with negative impact on overall treatment success, optionally due to high likelihood of increased muscle atrophy and/or stiffening of the shoulder.

10. The botulinum neurotoxin subtype E with reduced persistence for use in the treatment of claim 3, wherein said tendon-related disorder and/or injury is biceps tendon tear or retear.

11. The botulinum neurotoxin subtype E with reduced persistence for use in the treatment of claim 3, wherein said tendon-related disorder and/or injury is Achilles tendon tear or retear.

12. The botulinum neurotoxin subtype E with reduced persistence for use in the treatment of claim 3, wherein said tendon-related disorder and/or injury is tear of the flexor or extensor tendon of the underarm, hand or finger.

13. The botulinum neurotoxin subtype E with reduced persistence for use in the treatment of claim 3, wherein said tendon-related disorder and/or injury is a tendinopathy.

14. The botulinum neurotoxin subtype E with reduced persistence for use in the treatment of claim 13, wherein said tendinopathy is epicondylitis.

15. The botulinum neurotoxin subtype E with reduced persistence for use in the treatment of claim 14, wherein said epicondylitis is epicondylitis humeri radialis or epicondylitis humeri ulnaris.

16. A product comprising a botulinum neurotoxin subtype E with reduced persistence having a sequence according to SEQ ID NO: 1 or SEQ ID NO: 2, or a functionally active variant thereof, for use in the treatment of a patient, wherein the patient has experienced a tendon-related disorder and/or injury.

17. A method for treating a patient that has experienced a tendon-related disorder and/or injury comprising administering an effective amount of a botulinum neurotoxin subtype E with reduced persistence having a sequence according to SEQ ID NO: 1 or SEQ ID NO: 2, or a functionally active variant thereof.
